(19) United States
(12) Patent Application Publication
(10) Pub. No.: US 2010/0041056 A1
(43) Pub. Date: Feb. 18, 2010

(54) TEMPERATURE CONTROLLED NUCLEIC-ACID DETECTION METHOD SUITABLE FOR PRACTICE IN A CLOSED-SYSTEM

(75) Inventors: Paul Kinnon, Solana Beach, CA (US); David J. Saul, Auckland (NZ)

Correspondence Address: MORRISON & FOERSTER LLP, 425 MARKET STREET, SAN FRANCISCO, CA, 94105-2482, US

(73) Assignee: ZyGEM Corporation Limited, Hamilton (NZ)

(21) Appl. No.: 12/541,837

(22) Filed: Aug. 14, 2009

Related U.S. Application Data
(60) Provisional application No. 61/089,001, filed on Aug. 14, 2008.

(51) Int. Cl. C12Q 1/68

(52) U.S. Cl. 435/6

(57) ABSTRACT

The invention relates to a method that utilizes thermophilic proteases for the treatment of nucleic acids in a closed-system to be used in tandem with methods for the rapid detection of target nucleic acids present in a sample. These combined methods enable simplified, temperature-controlled devices to be used for accurate, streamline testing at the point of care for a wide variety of applications in the medical, industrial, environmental, quality control, security and research fields.

### CROSS-REFERENCE TO RELATED APPLICATIONS

This application claims the benefit of U.S. Provisional Application 61/089,001 filed on Aug. 14, 2008, the entire contents of which is hereby incorporated by reference. This patent application is related to U.S. 61/019,809; U.S. 61/038,389; U.S. Ser. No. 10/477,422; U.S. Ser. No. 11/640,495; WO 05/127,709 and WO 08/013,462.

### FIELD

This application relates generally to a method that can be practiced within a device for the rapid detection of target nucleic acid in a sample. More specifically, it relates to a nucleic acid treatment method that is compatible with temperature-controlled detection methods which can be used in a closed-system device for the detection of target nucleic acids present in a sample.

### BACKGROUND

Portable devices that accurately and robustly detect nucleic acids in a sample are desirable for a variety of medical, industrial, environmental, security, research and quality control purposes. Preferably, such devices are also rapid, yield accurate results and operate using a closed-system, i.e. a system that does not need to be opened during the course of the analysis in order to prevent yield reduction or accidental contamination with unwanted nucleic acids or nucleases. Nucleic acid detection strategies can be split into three stages: nucleic acid treatment, signal amplification and signal detection/analysis. A primary difficulty with these stages is that they generally require different devices to perform them. Therefore any automated beginning-to-end device requires different instrumentation for each stage in addition to a method for transferring the material from one internal instrument to the next. The need to combine the technology for each stage complicates the design for miniaturisation.

In regards to the nucleic acid treatment step, nucleic acid-based diagnostic procedures often require nucleic acid preparation from natural substances. Applications range from forensic DNA-fingerprinting to medical, agricultural and environmental monitoring. It is important that the nucleic acid treatment be free from contamination, particularly where the concentration of nucleic acid in the initial sample is very low or where contamination can lead to incorrect outcomes. This is particularly the case in forensic and evidence analyses where quantities of starting material may be on the order of picograms or less. Standard nucleic acid treatment techniques are problematic as the sample tube may require opening and shutting at stages throughout the preparation procedure, where contamination may occur simply as a result of the sample tube being opened to the atmosphere or being touched by a technician. Because of the ease with which a sample can be contaminated, it is preferred that reproducible nucleic acid treatment techniques utilize protocols directed to minimising such contamination.

After a nucleic acid has been treated to minimize contaminants, it can be subjected to nucleic acid detection methods to determine if a target nucleic acid is present in a sample. Many situations arise where it is desirable to detect low levels of specific nucleic acid sequences within the context of a complex mixture. A method intended for this purpose must be highly specific and sensitive. No simple method currently exists that can directly detect a single nucleic acid molecule of a specific sequence, and so all currently employed methods include a step or steps which amplify the signal. The most widespread method used to achieve this goal is the polymerase chain reaction (PCR). This method provides exponential amplification of target molecules by using thermal cycling and a thermostable DNA polymerase.

Current PCR technology used for the amplification of signal often necessitates lengthy purification procedures involving long incubations with proteinases, phenol/chloroform extractions and a finally an ethanol salt precipitation step before PCR can be conducted. Additionally, DNA purification protocols involving cells often involve incubating samples with Proteinase K and detergents, causing lysis of cells at temperatures where deleterious enzymes are released from cells that may degrade sample DNA and interfere with detection of target nucleic acid sequences. PreTaq™ was commercially available as a thermostable alternative to Proteinase K to clean up DNA without degradation, however the temperature-activity profile of PreTaq™ is not ideal as it remains active and is not readily removed at high temperatures and thus itself becomes a contaminant. It would therefore be advantageous to develop a protocol enabling simple, closed-tube reactions minimising the likelihood of contamination and removing the use of Proteinase K and other substances that may interfere with the PCR.

An inherent complication in this method is the requirement for the repeated cycling of the reaction between high and low temperatures. Thus, the method requires equipment that is more difficult to miniaturize. In response to this limitation, much effort has been expended to develop single-temperature, or isothermal, equivalents of PCR. One approach has been to use a polymerase that simultaneously achieves strand-displacement and strand-synthesis, thereby removing the need for the high-temperature step to produce single stranded DNA in traditional PCR methods.

There is clearly a need for an accurate, rapid and accessible method to identify target nucleic acid, particularly those corresponding to microorganisms, encountered in a wide range of situations or in mixed populations. The terms “target region”, “target sequence”, “target nucleic acid”, “target nucleic acid sequence”, “target polynucleotide”, and “target polynucleotide sequence” and grammatical equivalents thereof refer to a region of a nucleic acid which is to be detected. The term “target nucleic acid” or “target nucleic acid sequence” as used herein therefore includes the target nucleic acid to be detected, for example that present in a sample.

Essentially, the three stages in the nucleic acid detection process are preparation or treatment of the nucleic acid, amplification of a single indicating the presence of the target nucleic acid in a sample, and detecting the single produce by the presence of the target nucleic acid in the sample. Current strategies for each of these stages require different instrumentation and so multiple units must be incorporated into a miniaturised device and the materials must be transferred between these units by either hydrostatic or electromotive forces. Preferably each step would be simplified and be suitable for practice in a closed-system where all the stages can be processed in the same unit under compatible buffer conditions thus limiting complexity, cost, contamination and streamlining nucleic acid detection.

### SUMMARY OF PREFERRED EMBODIMENTS

A method for detecting target nucleic acid in a sample that is suitable for use in a temperature controlled device is described herein. The method includes i) treatment of nucleic acid in a sample, ii) production of a single indicating the presence of the target nucleic acid in a sample, and iii) detecting the single produce by the presence of the target nucleic acid in the sample.

In a first aspect, an embodiment provides a method for the detection of a target nucleic acid in a sample, the method including:
a. treating a sample with a thermophilic proteinase to prepare a target nucleic acid for detecting,
b. providing detection reagents that produce a signal indicating the presence of the target nucleic acid in the sample, and
c. detecting the signal to determine the presence of the target nucleic acid,

the steps i), ii) and iii) are advantageously performed in a single vessel or tube.

In one embodiment, the vessel or tube is a device. In a further embodiment, the device is a hand-held device.

In one embodiment, one or more steps i), ii) or iii) are temperature controlled.

In one embodiment, the thermophilic proteinase is EA1.

In one embodiment, step a) is performed at a temperature of about 65-80° C. for a time sufficient to digest protein.

In a further embodiment, step a) further includes incubating the thermophilic proteinase at a temperature at or above about 90° C. for a time that is sufficient to inactivate the proteinase.

In one embodiment, the method further includes the steps of:
a. treating the sample with a mesophilic enzyme, and
b. incubating the sample at a temperature below about 40° C. for a period of time that is sufficient to effect removal of cell walls from cells.

In a further embodiment, the mesophilic enzyme is a cellulase.

In one embodiment, the signal is fluorescence.

In one embodiment, the detecting is by PCR detection methods. In a further embodiment, the PCR detection method is real-time PCR.

In one embodiment, the detecting is by isothermal detection methods. In a further embodiment, the isothermal detection methods is by strand displacement amplification, rolling circle amplification, loop-mediated isothermal amplification, isothermal chimeric primer-initiated amplification of nucleic acids, Q-beta amplification systems or OneCutEventAmplificatioN. In yet a further embodiment, the isothermal detection methods utilizes Nuclease Chain Reaction (NCR), RNAse-mediated Nucleases Chain Reaction (RNCR), Polymerase Nuclease Chain Reaction (PNCR), RNAse-Mediated Detection (RMD), Tandem Repeat Restriction Enzyme Facilitated (TR-REF) Chain Reaction or Inverted reverse Complement Restriction Enzyme Facilitated (IRC-REF) Chain Reaction.

In one embodiment, the detection reagents are provided by microfluidics or a solid dispenser.

In one embodiment, the detection reagents are provided by microcapsules. In a further embodiment, the microcapsules are pre-disposed in the vessel or tube. In yet a further embodiment, the microcapsules are heat-labile capsules. In a further embodiment, the heat-labile capsules are agarose or wax beads. In yet another embodiment, the heat-labile capsules released the detection reagents at temperatures above the preferred incubation temperature used in the extraction step.

In one embodiment the detection reagents are resistant to the treatment process, in particular any enzymes required for the detection steps are resistant to proteolytic cleavage by the proteinase present for the purpose of preparing the nucleic acid from the biological material.

In one embodiment, the detection of the target nucleic acid is automated.

In one embodiment, the sample is blood, urine, saliva, semen, stool, tissue, swabs, tears or mucus.

In another embodiment, the sample is bacteria, fungi, archaea, eukarya, protozoa or virus.

In a further embodiment, the device or components of the device are disposable.

In a further embodiment, the device comprises an inlet port, an outlet port, a chamber, a detector for emitted fluorescence and an excitation light source.

In a further embodiment, the device further comprises microfluidics, microchips, nanopore technologies and miniature devices.

### BRIEF DESCRIPTION OF THE FIGURES

FIG. 1. Overview of nucleic acid detection strategies within a temperature controlled device.

FIG. 2. Single chamber nucleic acid treatment and detection using sequential liquid delivery of reagents.

FIG. 3. Single chamber nucleic acid treatment and detection using encapsulated reagents.

FIG. 4. Tube-based nucleic acid treatment and detection using encapsulated reagents.

FIG. 5. Real-time PCR traces where the treatment and detection steps were performed in the same closed tube.

FIG. 6. C_{T} values obtained in a qPCR reaction for different cell counts when DNA extraction and qPCR are performed in a single vessel.

### DETAILED DESCRIPTION

Nucleic acid detection strategies can be split into three stages: nucleic acid treatment, signal amplification and signal detection/analysis. Therefore, any fully automated nucleic acid detection device requires different instrumentation for each stage, and a method of transferring the material from one internal instrument to the next, complicating the design for miniaturization. The thermophilic proteinase nucleic acid treatment method disclosed herein is temperature modulated as are all amplification methods, whether isothermal or cycling. In addition, the conditions required for the thermophilic treatment are compatible with those for most amplification processes. Because of these factors, a device can be simplified to no more than a vessel with a heating/cooling mechanism to process raw sample material and take it all the way to a detectable signal. The inclusion of a detector is also facile. Hence the currently disclosed method enables devices with no pumps or need for microfluidics, however these can be used for more complex downstream applications if required.

Herein, a method for nucleic acid treatment, signal amplification and detection is described which can be practiced in closed-system devices that utilize heat-controlled reaction chains. The devices may be portable. Thermostable proteinases are used to prepare nucleic acid in a sample in tandem with nucleic acid identification techniques including PCR or isothermal detection methods. Heat control using either temperature dependent enzyme mixtures or temperature controlled release of encapsulated reagents simplifies the design of current nucleic acid diagnostic devices. Reducing complexity can reduce associated failure rate and cost. These techniques have the added benefit of being amendable to multiplexing for the simultaneous identification of multiple target nucleic acids in a mixed sample.

The term “treatment” used throughout the application refers to the process of increasing the availability of nucleic acid within a sample for processing by other manipulations. Implicit in the concept of “treatment” is that the nucleic acid is sufficiently free of interfering substances such as inhibitors, nucleases, other enzymes and nucleoproteins that it is effective in other manipulation methods. It is understood that the nucleic acid is not necessarily purified away from non-interfering compounds as to do so serves no purpose in the present device. The nucleic acid treatment minimizes the negative effects of interfering compounds.

The terms “nucleic acid”, “nucleic acid sequence”, “polynucleotide(s),” “polynucleotide sequence” and equivalents thereof as used herein mean a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. There is no intended distinction in length between the terms “nucleic acid”, “oligonucleotide” and “polynucleotide”, and these terms will be used interchangeably.

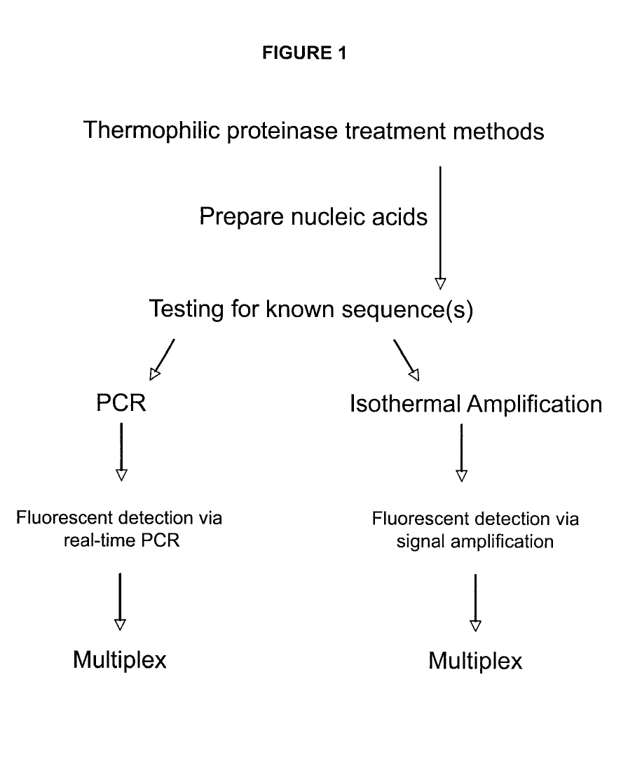

The method detailed in this disclosure is outlined in FIG. 1. In a first step, a thermostable proteinase such as EA1 is added to a sample to digest contaminating proteins at a temperature optimal for thermostable proteinase activity.

Samples can be obtained from a wide range of substrates including clinical, food and beverage or environmental samples. Typically, microbial samples are obtained from environmental sources and for food testing by either taking a sample of a liquid or solid or by swabbing a solid surface. Conveniently, clinical samples may be taken from tissues, blood, serum, plasma, cerebrospinal fluid, urine, stool, semen, swabs or saliva. Tissue samples may be obtained using standard techniques such as cell scrapings or biopsy techniques to collect animal tissue. Similarly, blood sampling is routinely performed, for example for pathogen testing, and methods for taking blood samples are well known in the art. Likewise, methods for storing and processing biological samples are well known in the art. For example, tissue samples may be frozen until tested. In addition, one of skill in the art would realize that some test samples would be more readily analyzed following a fractionation or purification procedure, for example, separation of whole blood into serum or plasma components.

Initially, mesophilic enzymes may also be utilized to degrade cell wall proteins or other contaminants. The temperature can then be adjusted to inactivate the thermophilic proteinase while at the same time, in certain embodiments, release detection reagents contained in heat-labile materials. After the nucleic acid has been prepared from the sample and the proteinase has been inactivated, the nucleic acid is combined with detection reagents customized for the detection of known nucleic acid sequences.

Known nucleic acid sequences can be detected by fluorescence using traditional PCR or isothermal signal amplification methods. Unlike PCR, isothermal signal amplification does not require temperature cycling. Both PCR and isothermal detection methods can be multiplexed for the simultaneous detection of multiple target sequences of interest.

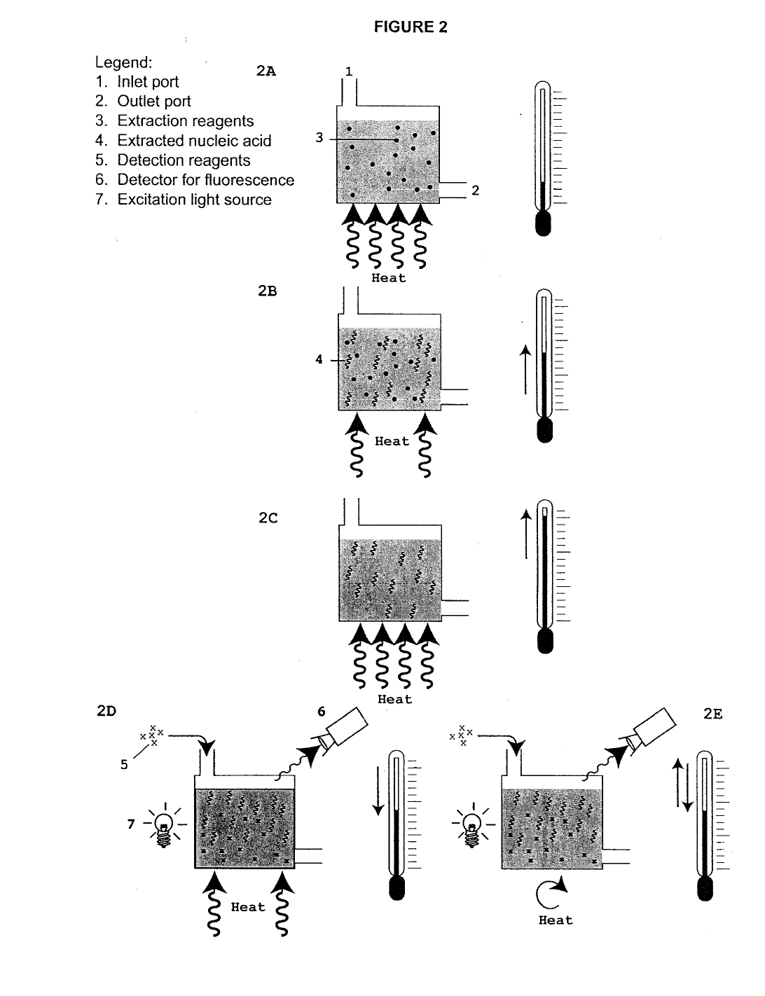

In a preferred embodiment, the method is occurring in a device. FIGS. 2-4 illustrate various examples of how the methods can be practiced in the context of a device. The preferred device would be portable and would allow for closed-system reactions, thus requiring little more than simple physical modulation of a reaction between sample insertion and result generation. Preferably, temperature is used to initiate and stop sequential chemical reactions allowing multi-step procedures to be performed without complex pumps, valves or microfluidics. Heat can be controlled by many simple devices including microelectronics, LEDs, Peltier plates or an incandescent light bulb.

A preferred embodiment for such a device would have compatible reaction conditions for all stages of the process, from nucleic acid treatment to signal amplification to signal detection. This detection system can be integrated with existing technologies that are specifically designed for buffer compatibility.

In a preferred embodiment, the device includes a single chamber. In another embodiment the chamber holds an externally supplied tube for example a PCR tube, which is placed within the device. In a further embodiment, the device comprises an inlet port, an outlet port, a chamber, a detector for emitted fluorescence and an excitation light source.

In other embodiments, the device further includes microfluidics, microchips, nanopore technologies and miniature devices. The device or components of the device may be disposable.

It should be appreciated that the present described devices are methods may have applications for a range of nucleic acid diagnostic techniques where clean-up of nucleic acids to remove contaminants is particularly beneficial, or for diagnostic techniques where the present devices and methods may be adapted to achieve a similar beneficial outcome.

### Thermostable Proteases for Nucleic Acid Treatment

As will be apparent to persons skilled in the art, samples suitable for use in the methods described herein may be obtained from the environment (such as soil, rock, water and plant material samples, for example) or from subjects, including tissues or fluids from a subject, so that the sample contains the nucleic acid to be tested.

Thermostable proteinases are added to the sample. Thermostable proteinases include proteinases that have protein degradation activity at high temperatures. Exemplary but not limiting, EA1 proteinase has been identified by the applicants as a preferred thermostable proteinase that is easier to remove at high temperatures. The sample is then incubated and subjected to a temperature shift. Following the temperature shift, protein degradation occurs. The procedure operates at 65-80° C. as these enzymes are highly active between these temperatures. At this temperature, the cells are lysed and the proteinases degrade contaminating protein. By way of example, they rapidly remove DNA-degrading nucleases at temperatures where these nucleases are inactive, thereby minimising degradation of the target nucleic acid sample.

While in preferred embodiments of the current disclosure a thermophilic proteinase is used, it is anticipated that thermophilic enzymes other than proteinases could also be used. For ease of reference throughout the specification, the thermophilic enzyme will herein be referred to as a proteinase. However, this should not been seen as a limitation for other enzymes that could also conceivably be used.

Mixtures of mesophilic enzymes active at lower temperatures and one of the above mentioned proteinases can be used initially to weaken and/or remove cell walls from plant, fungal tissue, bacteria, spores and biofilms before continuing with the closed-system procedure.

The practice of the disclosed method within a device relies on the proteinase and/or a proteinase/cell-wall degrading enzyme having differential activities at different temperatures. By cycling through the variable temperatures, the activities of different enzymes can be brought into play without the need for opening the system to add new reagents.

For applications that require low temperature digestion of nucleic acids (for example, restriction enzyme digestion of DNA), a proteinase that has very low activity at 37° C. need not be removed or inactivated. Where multi-step or multi-enzyme reactions are required, the proteinases can be used in an enzyme mixture. As there is such low activity below 40° C., other enzyme reactions are able to occur in the presence of the proteinases.

According to one aspect of the current disclosure, there is provided a method for the treatment of nucleic acid samples in a closed-system, including the steps of:
1) adding at least one thermophilic proteinase to a sample containing nucleic acid for testing, and
2) incubating the sample for a preferred period of time at 65-80° C. as required to effect one or more of the lysis of cells, digestion of proteins and digestion of cell-wall enzymes, where the thermophilic proteinase is stable and active at 65-80° C. but is inactivated and/or denatured when the sample is incubated at or above 90° C. without requiring the addition of further denaturing agents.

In preferred embodiments, the proteinase source includes Bacillus sp. strain EA1 being a neutral proteinase. The preferred characteristics for a thermophilic proteinase to be used within the proposed methods are that:
1) it is substantially stable and active within the range 65-80° C., and
2) it is able to be readily inactivated and/or denatured at or above 90° C., and
3) optionally it has a temperature-activity profile such that it has low activity below 40° C. such that accompanying mesophilic enzymes, for example, are not degraded.

The preferred incubation temperature required to affect one or more of the lysis of cells, digestion of proteins, digestion of cell-wall enzymes, via activity of the proteinase is 75° C. The preferred incubation temperature required to effect inactivation and/or denaturation of the proteinase is 94° C. However, it should be appreciated that these temperatures are given by way of example only and are not meant to be limiting in any way. It is anticipated that the proteinases will have differing profiles for both enzyme activity and stability over a range of temperatures and that such enzyme dynamics would be known to a skilled artisan. It is also anticipated such enzyme profiles for the proteinases could be determined with minimal experimentation. According to another aspect of the disclosure there is provided a method for the treatment of nucleic acid samples as described above, the method including the initial steps of:
1) adding at least one mesophilic enzyme and at least one non-specific thermophilic enzyme to a sample containing nucleic acid for testing, and
2) incubating the sample for a preferred period of time below 40° C. as required to effect removal of any cell walls via activity of the mesophilic enzyme.

In preferred embodiments the mesophilic enzyme is a cell wall degrading enzyme. The preferred initial incubation temperature required to effect removal of any cell walls via activity of the mesophilic enzyme is 37° C. Once again, this should not be seen as a limitation in any way.

After the nucleic acid has been prepared and the proteinase has been inactivated, the sample can then be tested for target nucleic acids. Known nucleic acid sequences of interest can be detected by PCR-based detection methods or isothermal-based detection methods described below.

### Signal Production & Detection of Target Nucleic Acid

In one aspect of the current method practiced, PCR-based detection methods can be used to detect nucleic acid sequences of interest prepared by the treatment methods detailed above.

A “PCR reagent” refers to any of the reagents used for PCR, usually a set of primers for each target nucleic acid, a DNA polymerase (preferably a thermostable DNA polymerase), a DNA polymerase cofactor and one or more deoxyribonucleoside-5′-triphosphates (dDTP's) or similar nucleosides. Other optional reagents and materials used in PCR are described below.

A DNA polymerase is an enzyme that will add deoxynucleoside monophosphate molecules to (usually the 3′-hydroxy) end of the primer in a complex of primer and template, but this addition is in a template dependent manner. Generally, synthesis of extension products proceeds in the 5′ to 3′ direction of the newly synthesized strand until synthesis is terminated. Useful DNA polymerases include, for example, Taq polymerase, E. coli DNA polymerase I, T4 DNA polymerase, Klenow polymerase, reverse transcriptase and others known in the art. Preferably, the DNA polymerase is thermostable meaning that it is stable to heat and preferentially active at higher temperatures, especially the high temperatures used for priming and extension of DNA strands. More particularly, thermostable DNA polymerases are not substantially inactive at the high temperatures used in polymerase chain reactions as described herein. Such temperatures will vary depending on a number of reaction conditions, including pH, nucleotide composition, length of primers, salt concentration and other conditions known in the art.

Particularly useful polymerases are those obtained from various Thermus bacterial species, such as Thermus aquaticus, Thermus thermophilus, Thermus filiformis, and Thermus flavus. Other useful thermostable polymerases are obtained from various microbial sources including Thermococcus literalis, Pyrococcus furiosus, Thermotoga sp. And those described in WO-A-89/06691 (published Jul. 27, 1989). Some useful thermostable polymerases are commercially available, such as, AmpliTaq™, Tth, and UlTma™ from Perkin Elmer, Pfu from Stratagene, and Vent and Deep-Vent from New England Biolabs. A number of techniques are also known for isolating naturally-occurring polymerases from organisms, and for producing genetically engineered enzymes using recombinant techniques.

A DNA polymerase cofactor refers to a non-protein compound on which the enzyme depends for activity. Thus, the enzyme is catalytically inactive without the presence of cofactor. A number of materials are known cofactors including, but not limited to, manganese and magnesium salts, such as chlorides, sulfates, acetates and fatty acids salts. Magnesium chlorides and sulfates are preferred.

Also needed for PCR are two or more deoxyribonucleoside-5′-triphosphates, such as two or more of dATP, dCTP, dGTP and dTTP. Analogues such as dITP and 7-deaza-dGTP are also useful. Preferably, the four common triphosphates (dATP, dCTP, dGTP and dTTP) are used together.

The PCR reagents described herein are provided and used in PCR in suitable concentrations to provide amplification of the target nucleic acid. The minimal amounts of primers, DNA polymerase, cofactors and deoxyribonucleoside-5′-triphosphates needed for amplification and suitable ranges of each are well known in the art. The minimal amount of DNA polymerase is generally at least about 0.5 units/100 μl of solution, with from about 2 to about 25 units/100 μl of solution being preferred, and from about 7 to about 20 units/100 μl of solution being more preferred. Other amounts may be useful for given amplification systems. A “unit” is defined herein as the amount of enzyme activity required to incorporate 10 nmoles of total nucleotides (dNTP's) into an extending nucleic acid chain in 30 minutes at 74° C. The minimal amount of primer is at least about 0.075 μMol with from about 0.1 to about 2 μMol being preferred, but other amounts are well known in the art. The cofactor is generally present in an amount of from about 2 to about 15 mMol. The amount of each dNTP is generally from about 0.25 to about 3.5 mMol.

The PCR reagents can be supplied individually, or in various combinations, or all in a buffered solution having a pH in the range of from about 7 to about 9, using any suitable buffer, many of which are known in the art.

Other reagents that can be used in PCR include, for example, antibodies specific for the thermostable DNA polymerase. Antibodies can be used to inhibit the polymerase prior to amplification. Preferably, the antibodies are specific for the thermostable DNA polymerase, inhibit the enzymatic activity of the DNA polymerase at temperatures below about 50° C. and are deactivated at higher temperatures. Useful antibodies include monoclonal antibodies, polyclonal antibodies and antibody fragments. Preferably, the antibody is monoclonal. Antibodies can be prepared using known methods such as those described in Harlow et al., Antibodies: A Laboratory Manual, Cold Spring Harbor, N.Y. (1988).

Light emitting labels can be used in PCR and isothermal detection methods. Mechanisms by which the light emission of a compound can be quenched by a second compound are described in Morrison, 1992, in Nonisotopic DNA Probe Techniques (Kricka ed., Academic Press, Inc. San Diego, Calif.), Chapter 13. One well known mechanism is fluorescence energy transfer (FET), non-radiative energy transfer, long-range energy transfer, dipole-coupled energy transfer, and Forster energy transfer. The primary requirement for FRET is that the emission spectrum of one of the compounds, the energy donor, must overlap with the absorption spectrum of the other compound, the energy acceptor. Styer and Haugland, 1967, Proc. Natl. Acad. Sci. U.S.A. 98:719, incorporated herein by reference, show that the energy transfer efficiency of some common emitter-quencher pairs can approach 100% when the separation distances are less than 10 angstroms. The energy transfer rate decreases proportionally to the sixth power of the distance between the energy donor and energy acceptor molecules. Consequently, small increases in the separation distance greatly diminish the energy transfer rate, resulting in an increased fluorescence of the energy donor and, if the quencher chromophore is also a fluorophore, a decreased fluorescence of the energy acceptor. In the methods, the signal emission of label, preferably a fluorescent label, bound to the probe is detected.

Exposure of a detection sequence means the detection sequence is rendered accessible for detection, for example accessible for binding to a detection probe. Conversely, the terms “hidden” or “masked” and their grammatical equivalents mean that the element(s) in respect of which these terms are used is/are not accessible. For example, a detection sequence may be hidden or masked when bound to nucleic acid molecule other than a detection probe. The term “hybridisation” and grammatical equivalents refers the formation of a multimeric structure, usually a duplex structure, by the binding of two or more single-stranded nucleic acids due to complementary base pairing.

Because the treatment system uses only temperature control, a PCR can be performed in the same vessel as the treatment, and use the same instrumentation within the device. The PCR buffer and the treatment buffer are compatible in the preferred embodiment. Deoxyribonucleotides, divalent ions and oligonucleotide primers can be supplied alongside the treatment reagents because these are unaffected by the enzymes and the process used to treat the nucleic acids. Some DNA polymerases, for examples Taq DNA polymerase, are degraded by the thermophilic proteinase in the treatment reagents. Hence, post-treatment delivery strategies for the polymerase must be considered. Possible strategies are: (1) delivery of the polymerase and any other sensitive reagents after the treatment process is complete. This can be a delivery via an inlet port by microfluidics or a solid dispenser. (2) The polymerase and other sensitive reagents can be added into the treatment reagents in a protected form. This can be in the form of a bead or film with the sensitive reagents microencapsulate within. (3) The polymerase can be modified to protect it from the proteinase for example by the attachment of antibodies. (4) Novel polymerases can be used that are resistant to proteolytic cleavage.

Once the PCR reagents have been supplied, thermal cycling can be achieved using the same heating device and controller used in the treatment process. PCR reactions can be multiplexed to assay for several target nucleic acids simultaneously.

Another aspect of the disclosure is directed to isothermal detection methods to detect target nucleic acid, wherein the method relies on the target nucleic acid-dependent amplification of signal from a detectable label bound to a nucleic acid probe. Isothermal amplification can be by strand displacement amplification, rolling circle amplification, loop-mediated isothermal amplification, isothermal chimeric primer-initiated amplification of nucleic acids, Q-beta amplification systems or OneCutEventAmplificatioN.

Techniques that may be exploited during in isothermal amplification are Nuclease Chain Reaction (NCR), RNAse-mediated Nucleases Chain Reaction (RNCR). Both of these methods replace strand displacement with the selective degradation of one of the strands of DNA. The process can be initiated by using restriction endonucleases or RNAse H when one of the strands contains ribonucleotides. The Polymerase Nuclease Chain Reaction (PNCR) relies on nuclease cleavage in the presence of target DNA followed by an extension process using a DNA polymerase, RNAse-Mediated Detection (RMD) which is a method of strand degradation by RNAse H on DNA:RNA hybrids. RMD is an effective linear amplification system that is sometimes used in combination with other methods. Tandem Repeat Restriction Enzyme Facilitated (TR-REF) Chain Reaction or Inverted reverse Complement Restriction Enzyme Facilitated (IRC-REF) Chain Reaction are two variants of a method that relies on the cyclical production of a detector probe that contains tandem repeats. These repeats are copied by a DNA polymerase when a specific oligonucleotide trigger can act as a primer. Next, restriction endonucleases attack the newly formed double-stranded DNA and this releases the original primer and a second primer so that two new cycles can be initiated. Isothermal amplification reactions can be multiplexed to assay for several target nucleic acid sequences of interest simultaneously.

It will also be appreciated that some nucleic acids exist that possess “strand invasion” properties, whether such strand invasion results in the displacement of the complementary strand of the target nucleic acid and the formation of a target probe duplex, or the formation of a target probe triplex, without the target sequence first being single-stranded. Peptide Nucleic Acids (PNAs) and derivatives thereof may be capable of strand invasion, whereby probes currently disclosed containing target nucleic acid binding regions comprising PNAs can be used to detect target nucleic acid that has not been rendered fully single-stranded. The use of target-binding regions comprising PNAs is particularly contemplated in circular probes, where, prior to the formation of the target probe hybrid, the target-binding region of the probe may be substantially double-stranded.

As used herein, “target-binding domain” and its equivalent “target binding domain” refers to nucleic acid sequence present in a nucleic acid molecule that is sufficiently complementary to nucleic acid sequence present in the target nucleic acid to allow the hybridisation of the target-binding region and the target nucleic acid, and so to form a target probe hybrid.

In certain embodiments of the current disclosure, the methods for detecting target nucleic acids are reliant on detecting or measuring the signal from a label, preferably the light emission of a probe labelled with a light-emitting label. The term “label”, as used herein, refers to any atom, molecule, compound or moiety which can be attached to a nucleic acid, and which can be used either to provide a detectable signal or to interact with a second label to modify the detectable signal provided by the second label. Preferred labels are light-emitting compounds which generate a detectable signal by fluorescence, chemiluminescence, or bioluminescence. Still more preferred labels are light-emitting compounds the signal of which is diminished or rendered undetectable when in sufficiently close proximity to a masking group, for example, a quenching chromophore.

Alternative labelling systems can be also be used that demonstrate the cleavage of a label from moiety that can be bound to a solid matrix. An example would be a biotin label that could be bound to immobilised avidin and thus non-cleavage of the probe would bind a secondary label present on the other end of the probe. Such a method would have applications for dipstick-based detection. Yet more detection system may use labels that can be distinguished by nanopore technology. The methods described herein are applicable to the detection of probes labelled with a single label, although multiple labels may be employed. Detection of the cleaved probe occurs when the label, for example a fluorophore, is sufficiently removed from the masking group, for example a quencher, by the cleavage event, or the probe-denaturing process the cleavage event allows. This diminishes the interaction of the masking group and the label and so allows emission of the signal. As used herein, the term “masking group” means any atom, molecule, compound or moiety that can interact with the label to decrease the signal emission of the label. The separation of label and masking group resulting from the cleavage event or the probe-denaturing process the cleavage event allows in turn results in a detectable increase in the signal emission of the attached label. Depending on the label, signal emission may include light emission, particle emission, the appearance or disappearance of a colored compound, and the like.

Preferred light-emitting labels and masking groups that can interact to modify the light emission of the label are described below. The term “chromophore” refers to a non-radioactive compound that absorbs energy in the form of light. Some chromophores can be excited to emit light either by a chemical reaction, producing chemiluminescence, or by the absorption of light, producing fluorescence. The term “fluorophore” refers to a compound which is capable of fluorescing, i.e. absorbing light at one frequency and emitting light at another, generally lower, frequency.

The term “bioluminescence” refers to a form of chemiluminescence in which the light-emitting compound is one that is found in living organisms. Examples of bioluminescent compounds include bacterial luciferase and firefly luciferase. The term “quenching” refers to a decrease in fluorescence of a first compound caused by a second compound, regardless of the mechanism. Quenching typically requires that the compounds be in close proximity. As used herein, either the compound or the fluorescence of the compound is said to be quenched, and it is understood that both usages refer to the same phenomenon.

Many fluorophores and chromophores described in the art are suitable for use in the methods presently disclosed. Suitable fluorophore and quenching chromophore pairs are chosen such that the emission spectrum of the fluorophore overlaps with the absorption spectrum of the chromophore. Preferably, the fluorophore would have a high Stokes shift (a large difference between the wavelength for maximum absorption and the wavelength for maximum emission) to minimize interference by scattered excitation light.

Suitable labels which are well known in the art include, but are not limited to, fluoroscein and derivatives such as FAM, HEX, TET, and JOE; rhodamine and derivatives such as Texas Red, ROX, and TAMRA; Lucifer Yellow, and coumarin derivatives such as 7-Me2N-coumarin-4-acetate, 7-OH-4-CH.3-coumarin-3-acetate, and 7-NH2-4-CH3-coumarin-3-acetate (AMCA). FAM, HEX, TET, JOE, ROX, and TAMRA are marketed by Perkin Elmer, Applied Biosystems Division (Foster City, Calif.). Texas Red and many other suitable compounds are marketed by Molecular Probes (Eugene, Oreg.). Examples of chemiluminescent and bioluminescent compounds that may be suitable for use as the energy donor include luminol(aminophthalhydrazide) and derivatives, and Luciferases.

While in most embodiments it will be preferred that the detectable label be a light-emitting label and the masking group be a quencher, such as a quenching chromophore, other detectable labels and masking groups are possible. For example, the label may be an enzyme and the masking group an inhibitor of said enzyme. When the enzyme and inhibitor are in sufficiently close proximity to interact, the inhibitor is able to inhibit the activity of the enzyme. On cleavage or denaturation of the probe, the enzyme and inhibitor are separated and no longer able to interact, such that the enzyme is rendered active. A wide variety of enzymes capable of catalysing a reaction resulting in the production of a detectable product and inhibitors of the activity of such enzyme are well known to the skilled artisan, such as 13-galactosidase and horseradish peroxidise.

### Computer Related Embodiments

Device control can be achieved by standard electronic methods using hardware, software and firmware typical of thermal cycling devices. Likewise, any integrated detection system could use similar programmable devices.

Data produced by the detection device may range from a simple yes/no detection when the device is used for detecting a specific agent to real-time data where the time is measured for the signal to reach a pre-defined threshold thereby giving quantitative data. Similarly, electrophoretic data could be produced in the form the taken for peaks of fluorescence to reach a detector placed at a point along a capillary electrophoresis device.

Data analysis can be achieved using a computer program supplied to the device either via and external electronic port, wireless technology, an internal storage device or internal firmware. For simple purposes for example a device with a specific role of determining the presence or absence of a single target nucleic acid, reporting may be in the form of any visible indicator such as a light or and LCD or LED display.

Where data requires more complex analysis or a greater level of user input, the raw data, processed data or partially processed data can be transferred to an external computer via any form of removable storage device or a communications cable.

In certain embodiments, the results can utilize wireless technology to obtain data base information or use database information stored on the device that may aid in the identification of target nucleic acid present in the sample. Results can be binary, i.e. present or not present, or they can be quantitative or multivariate.

### EXAMPLES

Aspects of the present disclosure have been described by way of example only and it should be appreciated that modifications and additions may be made thereto without departing from the scope thereof as defined in the appended claims.

### Example 1

### A Single Chamber Device with Liquid Delivery of Reagents

FIG. 2 illustrates one embodiment of the present device that comprises single chamber treatment and detection using sequential liquid delivery of reagents. The shape of the container can be circular, square, triangular or any other useful shape with numerous ports if needed. The chamber can also be optimized for microfluidic samples or larger volumes depending on the application. In step 2A, treatment reagents and substrate are added to the reaction chamber. In step 2B, the reaction temperature is adjusted to suit the treatment reagents. Nucleic acids are released into solution. In step 2C, the temperature is raised further so that the treatment reagents are inactivated. Step 2D, where an isothermal amplification/detection system is detailed, a single temperature is used. Detection reagents are added to the chamber. The reaction temperature is adjusted to suit the detection reagents. Detection of a specific agent is performed at this stage, in this example by fluorescence. In another embodiment, such as illustrated in step 2E, detection reagents are added to the chamber. In this example, the reaction temperature is cycled as for a quantitative PCR. Detection of a specific agent is performed at this stage, in this example by fluorescence.

### Example 2

### A Single Chamber Device with Encapsulated Reagents

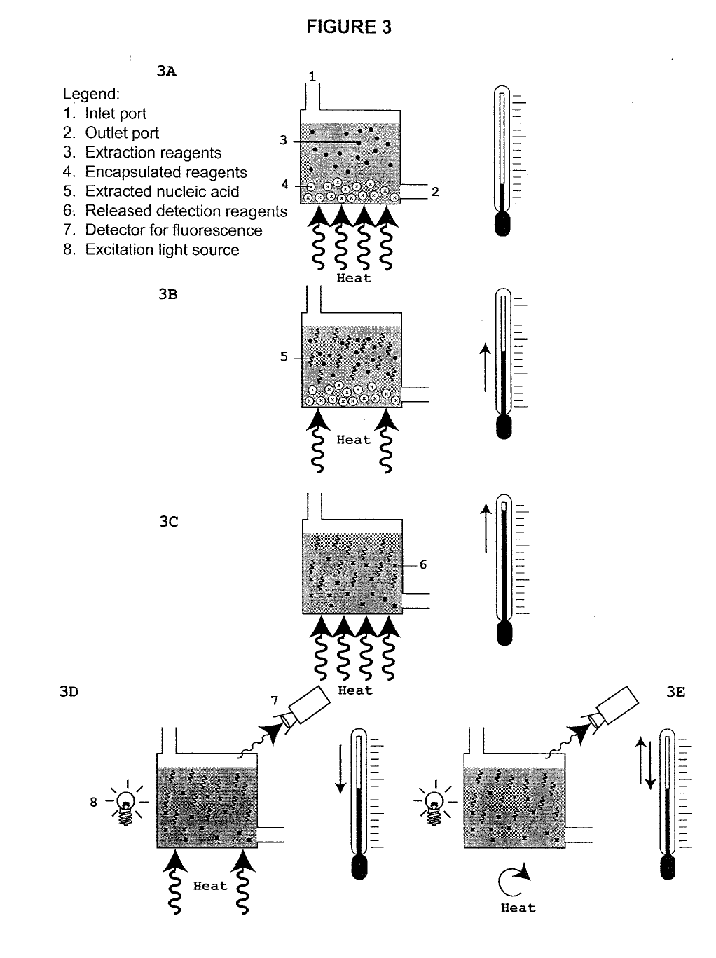

FIG. 3 shows a single chamber treatment and detection device that uses encapsulated reagents. In step 3A, treatment reagents and substrate are added to the reaction chamber along with the detection reagents but these are encapsulated to protect them from the proteinase used for treatment. In step 3B, the reaction temperature is adjusted to suit the treatment reagents. Nucleic acids are released. In step 3C, on completion, the temperature is adjusted so that the treatment reagents are inactivated while simultaneously, the detection reagents are released as the encapsulation bead melts. In step 3D, the reaction temperature is adjusted to suit the detection reagents. For an isothermal amplification/detection system, a single temperature is used. Detection of a specific agent is performed at this stage, in this example by fluorescence. In step 3E, in this example, the reaction temperature is cycled as for a quantitative PCR. Detection of a specific agent is performed at this stage, in this example by fluorescence.

### Example 3

### A Tube-Accommodating Device with Encapsulated Reagents

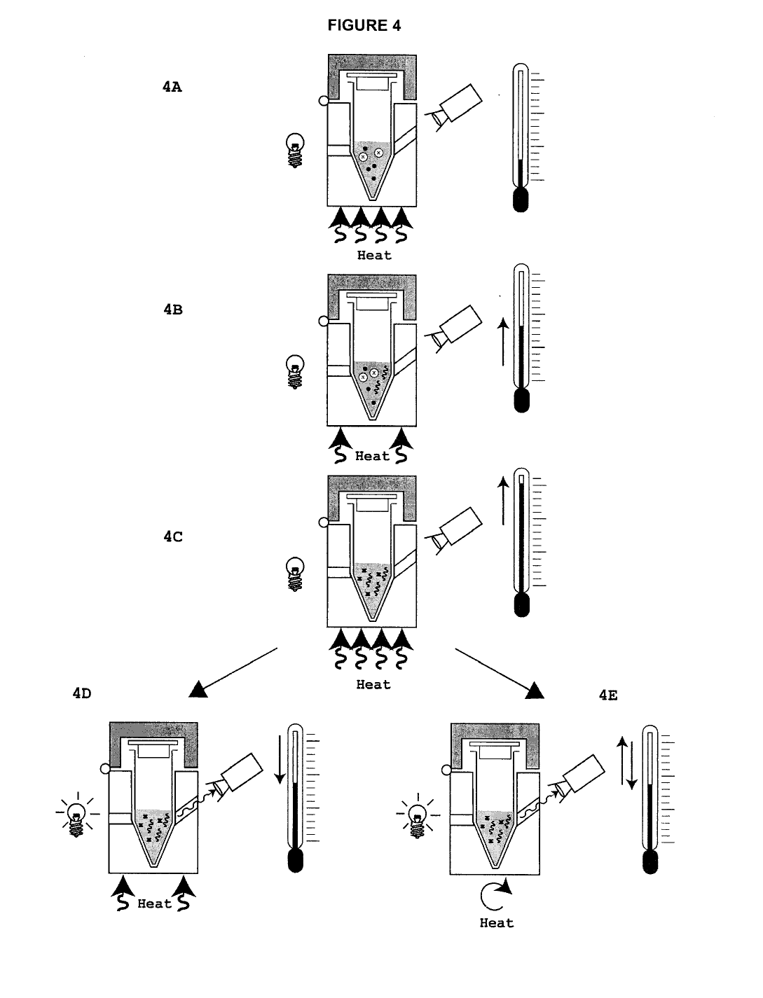

FIG. 4 illustrates a tube-based treatment and detection using encapsulated reagents. In step 4A, a tube containing treatment reagents, substrate and encapsulated detection reagents are is place in the device and covered. In step 4B, the reaction temperature is adjusted to suit the treatment reagents. Nucleic acids are released. In step 4C, on completion, the temperature is raised further so that the treatment reagents are inactivated while simultaneously, the detection reagents are released as the encapsulation bead melts. In step 4D, the reaction temperature is adjusted to suit the detection reagents. For an isothermal amplification/detection system, a single temperature is used. Detection of a specific agent is performed at this stage, in this example by fluorescence. In 4E, the reaction temperature is cycled as for a quantitative PCR. Detection of a specific agent is performed at this stage, in this example by fluorescence.

### Example 4

### Closed Tube Detection of Nucleic Acid from Buccal Cells

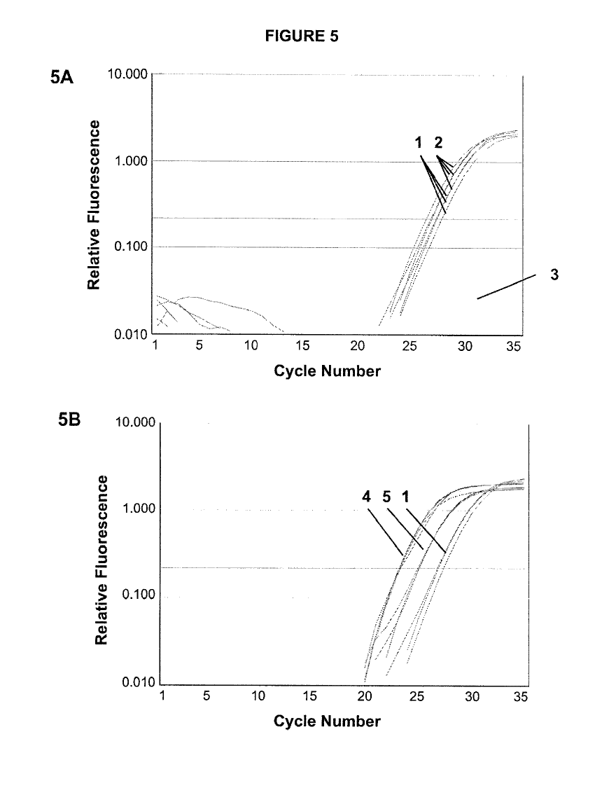

FIG. 5 details an experiment that demonstrates how a thermophilic proteinase can be used in combination with amplification and detection reagents in a single, closed vessel. In this example, all reagents, including untreated buccal cells, treatment reagents, amplification reagents and detection reagents were sealed in a 200 μl PCR tube and all processing was performed using only temperature to achieve nucleic acid detection from whole cells.

A buccal swab was taken from an individual following standard procedure. A standard cotton swab was used and the participant was instructed to rub the inside of the mouth and gums for 1 minute. Debris on the swab was suspended in 1 ml of 5 mM Tris (pH 8.3 at room temperature). The following cocktail of PCR and detection reagents was made. The primers were, Primer1: 5′-TCTCCTCCGATTTCAACAGTGA; Primer2, 5′-GGTCGTTGAGGGCAATGC. Platinum® Taq DNA Polymerase Invitrogen, San Diego, USA.

| Reagent | 1 reaction | 50 reactions |
|---|---|---|
| Water | 8.9 | 445 |
| Buffer | 2.5 | 125 |
| MgCl2 (supplied) | 0.75 | 37.5 |
| Primer1 (10 μM) | 0.5 | 25 |
| Primer2 (10 μM) | 0.5 | 25 |
| ROX 1 μM | 0.4 | 20 |
| SybrGreen (1/2500) | 0.5 | 25 |
| dNTP's (10 mM) | 0.5 | 25 |
| Platinum Taq (5 U/μl) | 0.2 | 10 |
| | 15 | 750 |

Using this master mix, the following cocktails were made. These are various combinations of reagents containing treatment reagents, whole cells or control DNA.

| | MM | Buccal | Human | EA1 | | |
|---|---|---|---|---|---|---|
| | as | cell | DNA | Proteinase | BSA | |
| | above | suspension | 0.25 ng/ul | 0.2 U/μl | 2.5 mg/ml | Water |
| 1 | 75 | | 5 | | 5 | 15 |
| 2 | 75 | | 5 | 5 | 5 | 10 |
| 3 | 75 | | | | 5 | 20 |
| 4 | 75 | 5 | | 5 | 5 | 10 |
| 5 | 75 | 5 | | | 5 | 15 |

Twenty five microliters of the five mixtures were dispensed into optically clear PCR tubes and sealed. All subsequent reactions were controlled by heat and no further tube openings. The tubes were heat cycled in an ABI 5700 Sequence detection system (Applied Biosystems, Forster City, USA) for 75° C. for 10 minutes (treatment step); 95° C. for 10 minutes (proteinase heat kill step and polymerase activation step); and 35 cycles of 95° C. for 30 sec, 60° C. for 30 sec, 72° C. for 30 sec with fluorescence measured in the last step (amplification/detection step).

The results in FIG. 5 demonstrate that cell treatment and detection can be performed under heat control when a thermophilic proteinase is used. The traces on 5A demonstrate that Platinum Taq DNA polymerase is resistant to hydrolysis by EA1 proteinase. The traces in 5B show the effect of the presence or absence of EA1 proteinase when whole cells are added to the mixture. When no proteinase is added (trace 5) the C_{T} value is approximately two cycles lower than when EA1 proteinase is present (trace 4). This equates to a quarter of the yield. Such a loss of yield is critical in trace samples.

FIG. 5 illustrates qPCR traces where the treatment reagents and the amplification and detection reagents are combined in a sealed tube. FIG. 5A shows traces produced where control DNA was added. FIG. 5B shows traces produced where whole cells were added (one control trace is included for reference). Sample 1 is the positive control (1.25 ng of purified human DNA). Sample 2 is a positive control that demonstrates that Platinum Taq is resistant to the proteolytic activity of EA1 proteinase. Sample 3 is the negative control (no trace). Sample 4 demonstrates that DNA can be prepared from human buccal cells in a closed tube with treatment, amplification and detection reagents present. Sample 5 shows the level of heat-mediated lysis of the buccal cells in the absence of the proteinase.

### Example 5

### Closed Tube Detection of Nucleic Acid from Bacterial Cells

The following experiment was performed on a dilution series of Escherichia coli cells and their presence was detected with universal 16S rRNA oligonucleotide primers. These primers are typical of the type used in microbial analysis. The following reagents and materials were used at the listed concentration were applicable: EA1 proteinase at 1 Unit per μl (ZyGEM Corporation Ltd); GIBCO UltraPure™ Distilled water (Invitrogen); Quanta Bioscience qPCR reagents; optically clear 96-well PCR plates (Axygen); maximum recovery filter tips (Axygen); and pPCR Primers at 10 μM:

| | |
|---|---|
| Forward: | GTCGTCAGCTCGTGTTGTGA |
| Reverse: | GCCCGGGAACGTATTCAC |

All work was performed in a PCR hood situated in an air-locked laboratory with positive air pressure generated through a HEPA filter and only previously unopened reagents, tubes, PCR plates, and filter-tips were used. Additionally, all surfaces were swabbed with 1% sodium hypochlorite prior to the experiment.

Escherichia coli MG1655 cells were grown overnight in LB broth. The cells were then centrifuged at 12,000 r.c.f for 5 minutes and resuspended in water to a cell density of 2×10⁷ per ml. This density is the equivalent of 10⁵ cells per 5 μl. A 1:10 serial dilution was made in ultrapure water wherein the lowest cell concentration was approximately 10 cells per 5 μl. Following the serial dilution, 5 μl of each dilution was placed into eight wells of an optically transparent 96-well microtitre plate. The following solution was added to four replicates:

| | |
|---|---|
| Water | 13 |
| Quanta PCR mix | 20 |
| Primer 1 10 μM | 0.8 |
| Primer 2 10 μM | 0.8 |
| EA1 proteinase @ 1 U/μl | 0.4 |
| Total volume | 35 |

In addition, four water controls were included for each reagent cocktail. The plate was then sealed with a transparent adhesive lid and held at 4° C. for 5 minutes in the dark after which time the plate was then exposed through the seal to a 600 W halogen lamp at 200 mm distant for 5 minutes with the tubes maintained at 4° C. This step is not necessary however can be useful for additional pre-treatments such as those described in U.S. Provisional Application Ser. No. 61/222,912. The samples were then placed in an Applied Biosystems 7300 Real-time PCR System and cycled as follows:

| | | | |
|---|---|---|---|
| DNA Extraction step: | | 75° C. 15 min | |
| Taq Activation step: | | 95° C. 5 min | |
| | | 95° C. 30 s | |
| PCR: | {open oversize brace} | 60° C. 30 s | x 45 |
| | | 72° C. 30 s (Fluorescence measured) | |

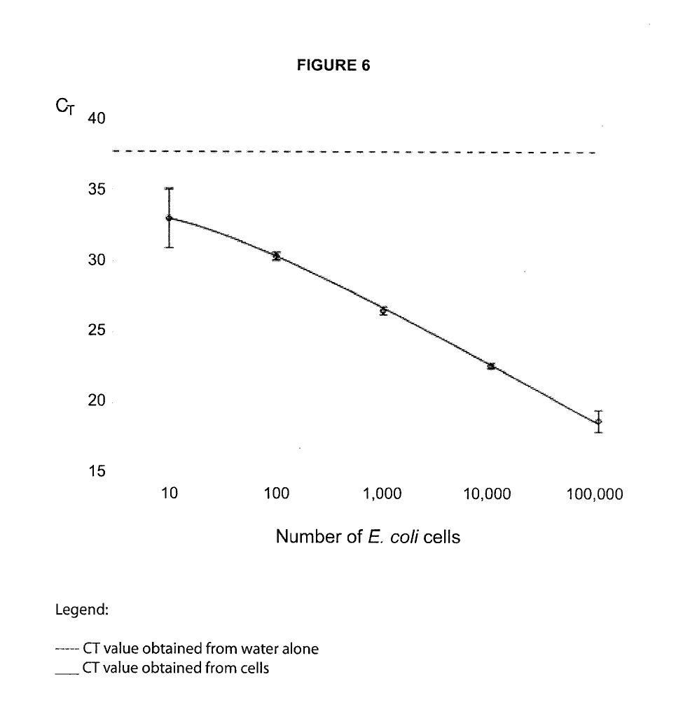

FIG. 6 shows a graph of the C_{T} values obtained for the closed vessel reaction. The C_{T} value is the number of PCR cycles that elapse before the threshold is reached. The higher the C_{T} value, the smaller the initial amount of DNA. The results clearly demonstrate that extraction and detection can be performed in a single reaction vessel without opening the tube.

What is claimed:

1. A method for the detection of a target nucleic acid in a sample, the method comprising:
i) treating the sample with a thermophilic proteinase to prepare the target nucleic acid for detecting,
ii) providing detection reagents that produce a signal indicating the presence of the target nucleic acid in the sample, and
iii) detecting the signal to determine the presence of the target nucleic acid,
wherein the steps i), ii) and iii) are performed in a single vessel or tube.

2. The method of claim 1, wherein the vessel or tube is a device.

3. The method of claim 2, wherein the device is a hand-held device.

4. The method of claim 1, wherein one or more steps i), ii) or iii) are temperature controlled.

5. The method of claim 1, wherein the thermophilic proteinase is EA1.

6. The method of claim 1, wherein step a) is performed at a temperature of about 65-80° C. for a time sufficient to digest protein.

7. The method of claim 6, wherein step a) further includes incubating the thermophilic proteinase at a temperature at or above about 90° C. for a time that is sufficient to inactivate the proteinase.

8. The method of claim 1, further comprising the steps of:
i) treating the sample with a mesophilic enzyme, and
ii) incubating the sample at a temperature below about 40° C. for a period of time that is sufficient to effect removal of cell walls from cells.

9. The method of claim 8, wherein the mesophilic enzyme is a cellulose or lysozyme.

10. The method of claim 1, wherein the signal is fluorescence.

11. The method of claim 1, wherein the detecting is by PCR detection methods.

12. The method of claim 1, wherein the PCR detection methods is real-time PCR.

13. The method of claim 1, wherein the detecting is by isothermal detection methods.

14. The method of claim 1, wherein the isothermal detection methods is by strand displacement amplification, rolling circle amplification, loop-mediated isothermal amplification, isothermal chimeric primer-initiated amplification of nucleic acids, Q-beta amplification systems or OneCutEventAmplificatioN.

15. The method of claim 1, wherein the isothermal detection methods utilizes Nuclease Chain Reaction (NCR), RNAse-mediated Nucleases Chain Reaction (RNCR), Polymerase Nuclease Chain Reaction (PNCR), RNAse-Mediated Detection (RMD), Tandem Repeat Restriction Enzyme Facilitated (TR-REF) Chain Reaction or Inverted reverse Complement Restriction Enzyme Facilitated (IRC-REF) Chain Reaction.

16. The method of claim 1, wherein the providing of detection reagents is by microfluidics or a solid dispenser.

17. The method of claim 1, wherein the providing of detection reagents is by microcapsules.

18. The method of claim 17, wherein the microcapsules are pre-disposed in the vessel or tube.

19. The method of claim 17, wherein the microcapsules are heat-labile capsules.

20. The method of claim 19, wherein the heat-labile capsules are agarose or wax beads.

21. The method of claim 20, wherein the heat-labile capsules release the detection reagents when exposed at a sufficient temperature to melt or dissolve the capsules.

22. The method of claim 1, wherein the detection reagents are resistant to proteolytic cleavage by the thermophilic proteinase.

23. The method of claim 1, wherein the detecting of the target nucleic acid is automated.

24. The method of claim 1, wherein the sample is blood, urine, saliva, semen, stool, tissue, swabs, tears or mucus.

25. The method of claim 1, wherein the sample is bacteria, fungi, archaea, eukarya, protozoa or virus.

26. The method of claim 2, wherein the device or components of the device are disposable.

27. The method of claim 2, wherein the device comprises an inlet port, an outlet port, a chamber, a detector for emitted fluorescence and an excitation light source.

28. The method of claim 2, wherein the device further comprises microfluidics, microchips, nanopore technologies and miniature devices.
